(19) United States
(12) Patent Application Publication
(10) Pub. No.: US 2025/0017512 A1
(43) Pub. Date: Jan. 16, 2025

(54) DEVICE AND METHOD FOR QUANTITATIVE ASSESSMENT OF SMALL NERVE FIBER FUNCTION

(71) Applicants: CONSEJO NACIONAL DE INVESTIGACIONES CIENTÍFICAS Y TÉCNICAS (CONICET), Ciudad Autónoma de Buenos Aires (AR); UNIVERSIDAD NACIONAL DE ENTRE RÍOS (UNER), Concepción del Uruguay Entre Ríos (AR)

(72) Inventors: José Alberto BIURRUN MANRESA, Oro Verde Entre Ríos (AR); Christian Ariel MISTA, Colonia Ensayo Entre Ríos (AR); Silvio Jorge LAUGERO, Paraná Entre Ríos (AR)

(21) Appl. No.: 18/710,865

(22) PCT Filed: Nov. 11, 2022
(86) PCT No.: PCT/IB2022/060878

§ 371 Date: May 16, 2024

(51) Int. Cl. A61B 5/377; A61B 5/00; A61B 5/372

(52) U.S. Cl. CPC A61B 5/377; A61B 5/372; A61B 5/4827

(57) ABSTRACT

The system disclosed herein provides selective Aδ and C small fiber stimulation by means of RF currents applied to the skin using small-area RF probes. The system also records body responses to said stimulation, for example, radiofrequency evoked potentials from the electroencephalogram, reaction times from a push-button and subjective pain ratings from a visual analog scale. These responses are analysed by an algorithm to provide an assessment of the small nerve fiber function. The device and methods may provide for selective small nerve fiber stimulation, followed by objective or subjective quantification of body responses to said stimulation. The device and methods might be used for several purposes, including determination of sensory detection thresholds and pain thresholds to thermal stimuli, objective characterization of pathological conditions involving nociceptive pathways, assistance in the diagnosis and prognosis of small fiber neuropathy, and assessment of efficacy and dose-response relationships of drugs for pain management.

### FIELD OF THE INVENTION

The present invention generally relates to energy emitting devices and methods for assisting in biological and medical assessment of thin nerve fiber function. The device and methods presented here might be used for several purposes, including determination of sensory detection thresholds (SDTs) and pain thresholds (PTs) to thermal stimuli, objective characterization of pathological conditions involving nociceptive pathways, assistance in the diagnosis and prognosis of small fiber neuropathy, and assessment of efficacy and dose-response relationships of drugs for pain management, among others.

### BACKGROUND OF THE INVENTION

The International Association for the Study of Pain defines pain as an unpleasant sensory and emotional experience associated with actual or potential tissue damage or described in terms of such damage. Many diseases are associated to the perception of pain, and in many cases, pain can be a pathology in itself. Therefore, understanding the mechanisms that generate and maintain pain is vital for the development of new diagnostic tests, therapies and drugs in order to improve the quality of life of people that suffer pain.

In normal conditions, pain perception starts with a potentially harmful stimulus, which is generally transduced into nociceptive information by high-threshold receptors in the peripheral nervous system called nociceptors. In general terms, there are four types of nociceptors: mechanical, thermal, polymodal and silent. Mechanical nociceptors respond to intense pressure, whereas thermal nociceptors respond to extreme temperatures (higher than 45° C. or lower than 5° C.). Both are grouped within the so-called mechano-thermo nociceptors and transmit nociceptive stimuli by thinly myelinated Aδ fibers. Polymodal nociceptors respond to nociceptive chemical, mechanical and thermal stimuli, and finally silent nociceptors are activated by chemical stimuli and can only respond to mechanical or thermal stimuli after they have been previously activated. These last two nociceptor types transmit nociceptive information by means of unmyelinated C fibers. The most important difference between the two fiber types is their conduction velocity (3 to 40 m/s for Aδ fibers and less than 3 m/s for C fibers), which is why fast, sharp and pricking sensations are associated with Aδ fibers and slow, burning sensations are associated with C fibers.

Nociceptive information is then transmitted from the periphery to the central nervous system through dorsal root ganglions to the spinal cord, through synapses in the superficial and deep dorsal horn. From here, nociceptive information ascends through spinothalamic and spinoreticular tracts to the brain. Nociceptive information activates several cortical and subcortical brain structures, from which the perception of pain emerges. A large number of studies indicate that brain responses to nociceptive information reflect the activity of a network of brain structures that is at least partially specific for nociception, which would potentially allow the identification of biomarkers that are specific for nociception and pain.

The most studied brain responses are evoked potentials, that are transient voltage changes in the brain's electrical activity in response to sensory stimuli, which may be recorded in an electroencephalogram. These voltage changes are positive and negative deflexions (peaks) in the electroencephalogram waves, and certain peak features, such as onset latencies and amplitudes, have been proposed as objective biomarkers for the assessment of the physiology and physiopathology of the nervous system [1,2]. As one of many examples, it has been shown that patients suffering from small fiber neuropathy present altered evoked potential features [3-5].

A key aspect in the assessment of the nociceptive system is the technology used for selective activation of small nerve fibers. The most used stimulus modality is thermal, and up to date, there are currently two main ways to deliver thermal stimuli that are appropriate for nerve fiber assessment. The first option is to use lasers that are specifically built for this purpose, which produce brief heat pulses with a fine-tuned control of the delivered energy, for example through fiber optics [1,6,7]. However, laser devices for neurology are rather expensive, and require certain safety measures, such as special safety equipment, warning lights to indicate laser light emission, a beam stop or attenuator, among others, that prevent its widespread use in the clinic. The second option for delivering thermal stimuli is through special Peltier-based thermodes for hot or cold stimulation, which is a common feature in thermal sensory analyzers [8,9]. These devices are also quite expensive, although unlike lasers, they do not require specific safety equipment, and thus, they are more frequently seen in the clinical practice. Nevertheless, its usefulness is limited because they have large contact areas and a slow temperature ramp, that causes unwanted jitter (i.e., loss of synchrony) in the evoked potential responses.

The alternative to thermal stimulation is electrical stimulation, in which small electrical currents (in the order of micro- or milliamps) are delivered to specific skin locations through surface electrodes to activate nerve fibers directly. However, surface electrical stimulation is in principle not selective for small nerve fibers, coactivating large myelinated Aβ fibers as well [7,10]. Recently, the use of thin needle intraepidermal electrodes has been proposed to overcome this problem. Research shows that intraepidermal electrical stimulation is able to selectively activate small nerve fibers by reducing the area of the electrical field generated by the stimuli, in a way that targets only the epidermis (where small nerve fibers are located) but does not penetrate the epidermis (where large myelinated fibers are) [11,12]. The disadvantages of intraepidermal electrical stimulation are that it is an invasive methodology (since the needle electrode does pierce the skin), the electrodes are not reusable and thus expensive, and electrical stimulation suffers from a phenomenon termed habituation, that is defined as a gradually diminishing response to repetitive stimulation on a fixed body site.

Therefore, there is still a need in the field for a device and a method to assess the function of small nerve fibers which overcome the shortcomings of the options already known in the art, as highlighted above.

### BRIEF DESCRIPTION OF THE INVENTION

The present invention describes a system for the assessment of small nerve fiber function, comprising
a) a radiofrequency (RF) generator configured to deliver radiofrequency (RF) currents;
b) small-area RF probes to apply the RF currents to a subject's skin;
c) a monitoring module for recording body responses to the RF currents applied by the RF probes; and
d) a microprocessor configured to: i) send stimulus features to the RF generator according to stimulation parameters selected by a user of the device, and ii) analyze the body responses to the RF currents recorded by the monitoring module to provide an assessment of the small nerve fiber function.

In an embodiment of the invention, the RF generator is configured by the microprocessor to generate RF currents of a duration ranging from 5 to 500 ms.

In another embodiment of the invention, the RF currents generated by the RF generator have a waveform selected from the group consisting of square-wave, linear, exponential, and bounded exponential. Preferably, the waveform of the RF currents is square-wave.

In another embodiment of the invention, the small-area probes are selected from contact probes and contactless probes. Preferably, the contactless probes comprise a distance-adjusting mechanism. In another embodiment of the invention, the body response recorded by the monitoring module is selected from the group consisting of radiofrequency evoked potentials from an electroencephalogram, reaction times from a push-button and subjective pain ratings from a visual analog scale. Preferably, the monitoring device is configured to record radiofrequency evoked potentials from an electroencephalogram.

It is another aspect of this invention to provide a method for diagnosing small fiber neuropathy, the method comprising the steps of:
1) providing a radiofrequency (RF) generator configured to generate RF currents;
2) applying small-area RF probes to a subject's skin, wherein the small-area probes are capable of delivering the RF currents to the subject's skin;
3) varying the RF stimulation intensity generated by the RF generator by means of an up-down staircase algorithm until a subjective sensory detection threshold is detected, wherein the detection of the subjective sensory detection threshold implies recording body responses from the subject by means of a monitoring module; and
4) comparing the obtained subjective sensory detection threshold with reference population values.

In a particular embodiment of this aspect of the invention, step 3) of the method comprises the sub steps of:
a. increasing the stimulation intensity until an upper threshold is reached; and
b. decreasing the stimulation intensity until a lower threshold is reached.

Preferably, the method comprises a step 3′, which comprises repeating sub steps a. and b. of step 3) a limited number of times to obtain a more precise estimation of the subjective sensory detection threshold.

In a particular embodiment of this aspect of the invention, the subjective sensory detection threshold is a pain threshold.

In another embodiment of this aspect of the invention, the small-area RF probes are contact probes, and are placed on the subject's skin.

In another embodiment of this aspect of the invention, the small-area probes are contactless probes, fixed at a determined distance from the subject's skin by means of a distance-adjusting mechanism. Preferably, the contactless probes are fixed at a distance of about 1 mm from the subject's skin.

In yet another embodiment of this aspect of the invention, the body responses from the subject recorded in step 3) are selected from the group consisting of radiofrequency evoked potentials from an electroencephalogram, reaction times from a push-button and subjective pain ratings from a visual analog scale. Preferably, the recorded body responses are radiofrequency evoked potentials from an electroencephalogram.

It is another aspect of the present invention to provide a method for diagnosing small fiber neuropathy, the method comprising the steps of:
1) providing a radiofrequency (RF) generator configured to generate RF currents;
2) applying small-area RF probes to a subject's skin, wherein the small-area probes are capable of delivering the electromagnetic pulses to the subject's skin;
3) varying the RF stimulation intensity generated by the RF generator by means of an up-down staircase algorithm until a subjective pain threshold is detected, wherein the detection of the subjective pain threshold implies recording radiofrequency evoked potentials from an electroencephalogram from the subject by means of a monitoring device, this step comprising the sub steps of:
   a. increasing the stimulation intensity until an upper threshold is reached; and
   b. decreasing the stimulation intensity until a lower threshold is reached;
4) repeating sub steps a. and b. of step 3) a limited number of times to obtain a more precise estimation of the subjective pain threshold;
5) selecting a suitable multiplier of the detection threshold, and delivering a number of consecutive radiofrequency stimuli at different spots in the application region on the subject's skin while recording the radiofrequency evoked potentials from the electroencephalographic signals;
6) detecting salient features from the radiofrequency evoked potentials by means of an automatic detection algorithm; and
7) comparing the detected features with reference population values.

### BRIEF DESCRIPTION OF THE DRAWINGS

FIG. 1. Block diagram of a system according to the invention.

FIG. 2. Diagram of the stimulus control and stimulation blocks of the RF generator of a system according to the invention.

FIG. 3. Average and standard deviation of electroencephalographic amplitudes at C3 channel after stimulation of the right arm.

FIG. 4. Average and standard deviation of electroencephalographic amplitudes at C3 channel after stimulation of the left arm.

FIG. 5. Average and standard deviation of electroencephalographic amplitudes at Cz channel after stimulation of the right arm.

FIG. 6. Average and standard deviation of electroencephalographic amplitudes at Cz channel after stimulation of the left arm.

FIG. 7. Average and standard deviation of electroencephalographic amplitudes at C4 channel after stimulation of the right arm.

FIG. 8. Average and standard deviation of electroencephalographic amplitudes at C4 channel after stimulation of the left arm.

### DETAILED DESCRIPTION OF THE INVENTION

The present invention relates to an energy emitting device and methods for assisting in biological and medical assessment of small nerve fiber function.

The present inventors have found that the application of currents to the skin in the radiofrequency (RF) spectrum (i.e., in the range of 200 kHz to 3.3 MHZ) is able to generate nociceptive-specific stimuli. At these frequencies, nerves and muscles can no longer be electrically excited, and the effects generated are due exclusively to tissue heating, so in strictly physiological terms, RF stimuli would be perceived and transduced as thermal stimuli, like those generated by laser-based devices.

The use of RF currents has been described for treating (not diagnosing) certain neuropathies, not related with small fiber neuropathies (SFN). U.S. Pat. No. 10,512,413 discloses a method for identifying and treating a neural pathway associated with chronic pain via nerve stimulation and brain wave monitoring of a mammalian brain including (1) positioning a probe to stimulate a target nerve, wherein the target nerve is suspected of being a source of chronic pain; (2) delivering a first nerve stimulation from the probe to the target nerve, wherein the first nerve stimulation is sufficient to elicit a chronic pain response in the brain; and (3) monitoring for evoked potential activity in the brain as a result of the first nerve stimulation. The method can also include delivering second and third nerve stimulations to confirm the correct identification of the neural pathway and to treat the chronic pain, respectively. However, the patent is directed to treat chronic pain (which is not an SFN), and ultra-high frequency electrical nerve stimulation (corresponding to frequencies in the RF range) is disclosed to ablate or otherwise sufficiently impair the target nerve which was previously detected by stimulation in other frequencies. Additionally, the stimuli are delivered by means of a percutaneous (i.e., invasive) probe.

The present inventors have found that the application of RF currents shows the ability to stimulate patients non-invasively and elicit brain radiofrequency-evoked potentials, that may be analyzed to detect the presence or absence of small fiber neuropathies (SFN) by quantifying components such as peak latency, wave amplitude and spatial topography of the brain response.

Therefore, the present invention provides a system for the assessment of small nerve fiber function, comprising:
a) a radiofrequency (RF) generator configured to generate thermal stimuli from electromagnetic pulses in the RF range;
b) small-area RF probes to apply the electromagnetic pulses to a subject's skin;
c) a monitoring device for recording body responses to the electromagnetic pulses applied by the RF probes; and
d) a microprocessor configured to: i) send stimulus features to the RF generator for converting them to thermal stimuli according to stimulation parameters selected by a user of the device, and ii) analyze the body responses recorded by the monitoring module to provide an assessment of the small nerve fiber function.

### RF Generator

The RF generator of the system of the invention must be able to generate thermal stimuli on the subject's skin by means of RF currents.

The system enables RF stimulation in brief pulses of different durations and shapes to selectively activate A or C fibers. The duration of the pulses ranges from 1 to 500 ms in 1 ms steps. The shape of the pulses (i.e., the waveforms of the pulses) may vary according to the specifications of the equipment used. For instance, the pulses may have square-wave, linear, exponential or bounded exponential shapes, among others. Preferably, the waveform of the pulses is selected from the group consisting of square-wave, linear, exponential and bounded exponential. More preferably, the waveform of the electromagnetic pulses is square-wave.

RF stimulation intensity is selected in an ad hoc scale, ranging from 1 to 100 arbitrary units (au), covering the range for the output power of the device. Output power, measured in watts (W), is approximately a linear function on the output impedance, measured in ohms (Ω). Impedance primarily depends on the characteristics of tissue being stimulated. Output power ranges approximately from 0.1 to 0.5 W for low skin impedance (e.g., 100Ω) and from 0.5 to 5 W for high skin impedance (e.g., 2000Ω).

### RF Probes

The probes comprised within the system of the invention are similar to existing electrocautery probes. Generally, they are thin metal rods of approximately 10-15 cm length attached to the system by a conductive wire.

RF stimulation can be performed in two modes: contact and spark. In contact mode, specific contact probes are placed on the target body region (e.g., hand or foot) in a way that the tip of the contact probe makes light contact with the skin, without exerting additional pressure. In spark mode, specific contactless probes are placed in close proximity to the target body region, generating a RF spark that is able to bridge the gap between probe and skin when said gap is sufficiently small to thus provide the desired stimuli.

The probes which may be used according to the invention have interchangeable tips of different shapes. For example, said shapes may be pin tip, flat tip, or ball tip. Each shape has a different surface contact area, thus eliciting slightly different sensations and effects after RF stimulation. Typically, the small-area RF probes comprised within the system of the invention have a skin surface contact area of 1 to 5 mm² when operating on contact mode.

When the stimulation is performed in contact mode, a novel kind of probe may additionally be used, which consists of a two-point pin tip for bipolar stimulation, in which the distance between tips is adjustable. This new kind of tip allows for an additional assessment of a two-point discrimination stimulus (i.e., the minimum distance at which two stimulated points feel like two separate stimuli and not as a single one).

When the stimulation is performed in spark mode, the contactless probes must include a distance-adjusting mechanism to ensure that the distance between the probes and the subject's skin is fixed at a determined value. Preferably, the contactless probes are kept at a distance of 1 mm from the subject's skin. This is particularly advantageous taking into account the non-invasiveness of probes which are not in contact with the subject's skin.

### Monitoring Device

The RF current applied to the subject's skin by means of the RF generator and the RF probes generate a series of body responses which are recorded by a monitoring module comprised in the system of the invention for further processing.

The monitoring module is configured to record the body responses, which may be obtained in different forms by various means. For instance, the recorded body responses may be radiofrequency evoked potentials from an electroencephalogram, reaction times from a push-button and subjective pain ratings from a visual analog scale.

In a preferred embodiment of the invention, the recorded body responses are radiofrequency evoked potentials from an electroencephalogram. In this configuration, the monitoring module records the electroencephalogram of the subject through surface electrodes placed in the scalp to measure the level of brain activity in plurality of different regions of the subject's brain, and it is synchronized with the RF generator to provide exact timing for the stimuli and their evoked responses.

In another embodiment of the invention, the recorded body responses are reaction times. In this configuration, the monitoring module records the motor response of the subject through a push button, and it is synchronized with the RF generator to provide exact timing for the stimuli and their motor responses.

In another embodiment of the invention, the recorded body responses are subjective pain ratings. In this configuration, the monitoring module records the subjective perceived intensity of the stimulus by the subject through a visual analog scale. The scale ranges from 0 to 100, in which zero means no perception and 100 is the tolerance limit.

### Microprocessor

The system includes a microprocessor which interprets the stimulation parameters entered by the user of the system and generates stimulus features which are delivered to the RF generator for transducing them into RF currents for application to the subject's skin.

The microprocessor also analyzes the body responses recorded by the monitoring module, to provide an assessment of the subject's small nerve fiber function, and thus diagnose small nerve neuropathies, if present. The analysis of the recorded body responses is performed by a custom-made algorithm that integrates and processes the information available and provides an assessment of the small nerve fiber function.

When the body responses are brain evoked potentials from an electroencephalogram (EEG), the microprocessor preferably uses an algorithm to analyze the data generated by the monitoring module which is selected from the algorithms described in Biurrun Manresa et al. (2015) “On the Agreement between Manual and Automated Methods for Single-Trial Detection and Estimation of Features from Event-Related Potentials.” PLOS ONE 10 (8): e0134127. doi: 10.1371/journal.pone.0134127, the contents of which are hereby incorporated by reference.

Briefly, the data analysis is preferably carried out using an algorithm based on the derivative of the signal that classifies using fuzzy logic (DRIV), or an algorithm based on wavelet filtering and multiple linear regression (WVLT). The implementation of the DRIV algorithm may be carried out in C++, which is freely available at https://sourceforge.net/projects/stfderp, whereas the MATLAB implementation of the WVLT algorithm is freely available at http://iannettilab.webnode.com. These two methods are readily available and represent two very different approaches to detection/estimation: the DRIV algorithm mimics the decision process performed by a human observer during a visual detection task, whereas the WVLT algorithm relies on a linear model of the filtered signal in order to estimate the amplitudes and latencies of the peaks at single-trial level.

Radiofrequency evoked potentials elicited by RF stimulation display two characteristic peaks: a negative deflection (N2) around 170 ms and a complex of positive waves, with a peak at approximately 250 ms (P2).

### DRIV Algorithm

The first derivative of the EEG signal is calculated using numerical differentiation, in order to detect all local maxima (for P2) and minima (for N2) in the evoked potential waveform. Maxima and minima are located in the points where the derivative of the signal changes its sign (from positive to negative or vice versa). All the local maxima/minima found in each trial are further weighted within three fuzzy zones defined for the N2 and P2 peaks. Each fuzzy zone has a central latency and two boundaries. The fuzzy weights are defined by two quadratic functions that depend on the corresponding peak latency found in the average evoked potential (central latency) and its expected variability (boundaries). Consequently, the fuzzy zones have a maximum weight at the central latency that decrease in a quadratic fashion towards the boundaries, and the weight is set to zero outside the boundaries. The weighting process is performed by multiplying the evoked potential amplitude of the maxima/minima with the weight given by the correspondent fuzzy zone. The resulting maximal values for each zone are then selected as peaks, under the condition that their amplitude values are negative (for N2) or positive (for P2).

### WVLT Algorithm

Initially, single-trial evoked potential signals are represented in the time-frequency domain using the continuous Morlet wavelet transform (CWT, bandwidth parameters f_{b}=0.05 and f₀=6) and squared to obtain the magnitude of their power spectrum. These representations are then averaged, resulting in a time-frequency matrix that is further thresholded to obtain a binary mask. This mask was applied to each single-trial time-frequency representations to filter out wavelet coefficients with low energy. The filtered single-trial evoked potentials in the time domain are then reconstructed by using the inverse continuous wavelet transform (ICWT). The automated detection of N2 and P2 amplitudes and latencies is performed using a multiple linear regression approach [4]. Two regressors per peak (signals in the time domain and their first derivative) are obtained for each subject from the filtered average evoked potential. In order to obtain the regressors for each peak, the average evoked potential is separated where the voltage signal equals zero. Each single-trial evoked potential is then fitted with the set of regressors obtained from the averaged evoked potential using a least squares approach. The single-trial amplitudes and latencies of the N2 and P2 peaks are then obtained from the fitted regressors by measuring the maximum voltage peaks within a time window centred on the latency of each average brain evoked potential peak previously determined.

### Diagnostic Method

The system of the invention is capable of applying RF currents to the subject's skin and recording and analyzing the body response thus generated to provide an assessment of the small nerve fiber function, which may be indicative of a small nerve neuropathy in the subject.

Therefore, it is another aspect of this invention to provide a method for diagnosing small fiber neuropathy by means of the system of the invention, the method comprising the steps of:
1) providing a radiofrequency (RF) generator configured to generate thermal stimuli from electromagnetic pulses in the RF range;
2) applying small-area probes to a subject's skin, wherein the small-area probes are capable of delivering the electromagnetic pulses to the subject's skin;
3) varying the RF stimulation intensity generated by the RF generator by means of an up-down staircase algorithm until a subjective sensory detection threshold is detected, wherein the detection of the subjective sensory detection threshold implies recording body responses from the subject by means of a monitoring module; and
4) comparing the obtained subjective sensory detection threshold with reference population values.

As previously disclosed, the RF generator may provide brief pulses of different durations and shapes to selectively activate A or C fibers. A typical stimulation configuration for A delta fiber activation would consist of 5 square-wave pulses. Each pulse would have 1 ms duration, and there would be 5 ms between adjacent pulses. Stimulation intensity would depend on the detection threshold and the pain threshold for RF stimulation, but it would commonly be between 30 and 40 au.

The advantage of this configuration for RF stimulation is that it allows for very brief stimuli with a very steep temperature increase rate, which allows for fast evoked responses (as short as 50 ms) to be recorded in the electroencephalographic signals.

The up-down staircase algorithm of step 3) is meant to determine a subjective sensory detection threshold for the subject. For instance, the subjective sensory detection threshold may be a pain threshold for the subject.

Both an upper threshold and a lower threshold may be detected as part of the determination of the subject's subjective sensory detection threshold. Therefore, in a particular embodiment of this aspect of the invention, step 3) of the method comprises the sub steps of:
a. increasing the stimulation intensity until an upper threshold is reached; and
b. decreasing the stimulation intensity until a lower threshold is reached.

The manner upon which the thresholds are detected depends on the body responses recorded by the monitoring module.

For instance, if the monitoring module records reaction times from a push-button, the stimulation intensity is first increased until the upper threshold is reached, signalled by an event triggered by the push-button, and then decreased until it falls below the lower threshold, signalled by the absence of events triggered by the push-button.

Similarly, if the monitoring module records subjective pain ratings from a visual analog scale, the intensity is first increased until the desired pain intensity is reached, signalled by reaching or exceeding the target pain intensity level on the visual analog scale, then decreased until it falls below the desired pain intensity signalled by falling below the target pain intensity level on the visual analog scale.

Preferably, the monitoring module records evoked potentials from the electroencephalogram of the subject. In this case, the RF generator is configured automatically through the microprocessor to vary the stimulation intensity until an objective evoked potential threshold is detected by means of an up-down staircase algorithm, in which the intensity is first increased until a brain evoked potential is detected from the electroencephalogram, and then decreased until a brain evoked potential can no longer be detected from the electroencephalogram.

In any of the aforementioned cases, repeating the sub steps of step 3) may provide a more precise estimation of the subjective sensory detection threshold. Therefore, preferably, the method comprises a step 3′, which comprises repeating sub steps a. and b. of step 3) a limited number of times to obtain a more precise estimation of the subjective sensory detection threshold. Preferably, sub steps a. and b. of step 3) are repeated three times.

As encompassed by the method of the invention, RF stimulation can be performed in two modes: contact and spark, as defined previously in this description.

Therefore, in an embodiment of this aspect of the invention, the small-area probes are contact probes, and are placed on the subject's skin. When the RF stimulation is carried out in contact mode, the probes are placed on the skin with minimal contact, without exerting any pressure thereon.

In another embodiment of this aspect of the invention, the small-area probes are contactless probes, fixed at a determined distance from the subject's skin by means of a distance-adjusting mechanism. Preferably, the contactless probes are fixed at a distance of about 1 mm from the subject's skin.

Once the subjective sensory detection threshold of the subject is obtained, the assessment of whether the subject has a small fiber neuropathy is done by comparison with reference population values. As a general consideration, thresholds in the extreme 5% values of the population distribution can be considered indicative of small fiber neuropathy.

An alternative method of for diagnosing small fiber neuropathy by recording radiofrequency evoked potentials which is also within the scope of the present invention involves additional steps after determining the subjective pain threshold of the subject.

In this alternative method, once the subjective pain threshold has been determined, a suitable multiplier thereof is selected, and a number of consecutive RF stimuli with an intensity according to the selected multiplier are delivered to the subject at different spots in the region of interest (e.g. hand of foot) while recording the radiofrequency evoked potentials from the electroencephalographic signals. Preferably, the multiplier is two times. Also preferably, the number of RF stimuli delivered is 30. Also preferably, the RF stimuli are spaced between 10 and 15 seconds.

Afterwards, the alternative method comprises detecting salient features from the brain evoked potentials by means of an automatic detection algorithm. Preferably, the automatic detection algorithm is selected from the DRIV and WVLT algorithms. Most preferably, the automatic detection algorithm is the DRIV algorithm.

Finally, the alternative method comprises comparing the obtained features with reference population values. Generally, features in the extreme 5% values of the population distribution can be considered indicative of small fiber neuropathy.

The advantages related to the presently described technology are significant. RF stimulation technology is non-invasive and safe, does not require additional equipment and can be used in most clinical environments, even in a doctor's office. Furthermore, the stimulation is selective for small nerve fibers (given that it generates a thermal stimulus) and it can be easily applied in multiple body sites using small-area probes, so it does not suffer from habituation.

### EXAMPLES

### Example 1—System According to the Invention

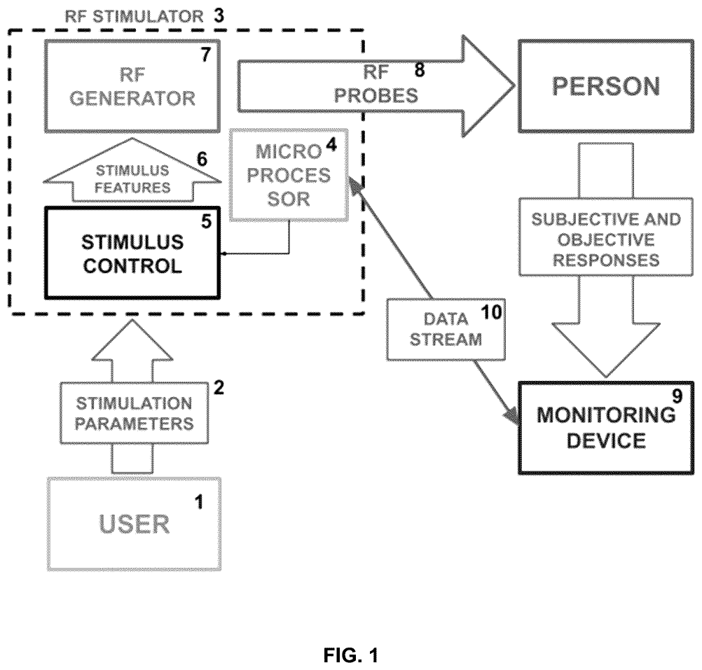

FIG. 1 illustrates an example of a system for quantitative assessment of small nerve fiber function, according to a non-limiting embodiment of the present invention. The system includes an RF generator 3, that generates thermal stimuli with specific parameters 2 set by the user 1. These parameters are interpreted by the microprocessor 4, that generate stimulus features 6 according to the desired stimulation parameters, which are sent to the stimulus control block 5, transduced into RF current by the RF generator 7 and applied to the skin using small-area RF probes 8. The system also records body responses to said stimulation through a monitoring module 9, for example, (but not limited to), radiofrequency evoked potentials from the electroencephalogram, reaction times from a push-button and subjective pain ratings from a visual analog scale. The data stream 10 generated by these responses is analysed by the microprocessor 4 using a custom-made algorithm that integrates and processes the information available and provides an assessment of the small nerve fiber function.

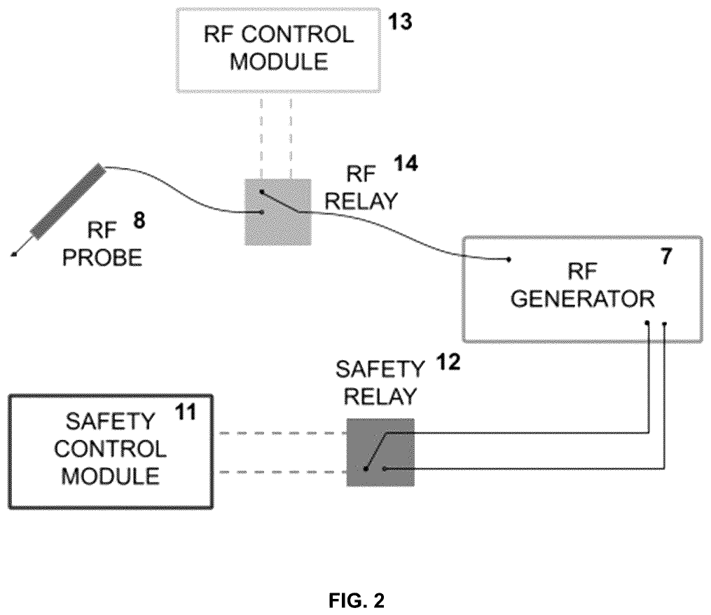

FIG. 2 provides details regarding the implementation of the RF generator 3. The stimulus control block 5 is implemented as two distinct modules, the safety control module 11 and the RF control module 13. Both modules command specific relays, the safety relay 12 and the RF relay 14. The safety relay ensures that stimulation will be interrupted in case the microprocessor fails, whereas the RF relay control the actual duration of the RF stimulus.

### Example 2—Experimental Protocol of a Study for Assessing SFN in a Subject

### 2.1. Participants

The experimental protocol comprised the participation of healthy volunteers, most of them selected from the student body and teacher staff of the Engineering School of the National University of Entre Rios, complying with the following inclusion criteria:
age between 18 and 60 years;
no history of neurological disease, chronic pain and musculoskeletal disorders;
will and capability of understanding completely the content and scope of the experiment, and to comply with the instructions thereof;
absence of fever, tuberculosis, malign tumours, infectious processes, or acute inflammatory processes;
no pacemakers or metallic prostheses;
no use of analgesics within 24 hours prior to the participation in the experiment.

### 2.2. RF Stimulation

RF stimulation was performed using a prototype of the device described in this patent. The stimulation features used were as follows: 40 stimuli of 25 ms length, applied with random intervals of 8 to 12 s, varying the location of the stimulation tips in a limited area to avoid habituation. Probes with a two-point pin tip were used, in bipolar mode, and applied to the internal zone of both forearms in supine position. Intensity was determined prior to the register, ensuring that the subject clearly feels 100% of the stimuli. To that end, a reduced number of stimuli were applied, increasing progressively their intensity until the intensity corresponding to the perception threshold was determined. To ensure a clear brain response, applied intensities were slightly higher than the perception threshold.

### 2.3. EEG Recording

For the EEG recording, five silver/silver chloride (Ag/AgCl) cup electrodes located in Cz, C3, C4, A1 (reference) and A2 (ground) were used, according to the standard 10-20 configuration. For the acquisition the OpenBCI amplifier was used, along with the OpenBCI GUI software to record the data. The amplifier was configured so that the resulting gain was 12. A sampling frequency of 2 kHz was used, as well as an 8^{th}-order Butterworth band-pass filters with cutoff frequencies at 0.5 and 100 Hz.

### 2.4. Experimental Session

The volunteers were received in a laboratory of the Engineering School, in a place with privacy to establish a comfortable communication. They were informed, in simple terms, the purpose of the project and the necessary measurements to participate therein.

In the beginning of each session, the volunteer was asked to sit comfortably. Next, the electrodes were applied, and it was verified that the signals were recorded correctly. The attention level has an important impact in the amplitudes of the evoked potentials, which is why the subject was asked to stay as attentive to the stimulus detection as possible. Headphones were then applied to the volunteer to isolate them from environmental sounds, to ensure that the evoked response obtained was mainly due to nociceptive stimuli and not by auditory stimuli.

Once the recording was complete, all electrodes were removed, after which the session was finished.

### 2.5. Pre-Processing and Statistical Analysis

For the processing of the obtained signals, two Matlab® toolboxes were used: EEGLAB (sccn.ucsd.edu/eeglab) y Letswave (www.letswave.org). Both are open-access, but they work exclusively with Matlab®. The raw signals were loaded with EEGLAB, and a “.set” file for Matlab® was generated with the identification of the events. Letswave was used to process the signals where, in first instance, a segmentation was carried out to generate epochs from −1 to 2 s with respect to the event. Afterwards, a baseline correction was performed, and the stimulus artifact from −1 to 30 ms with respect to the event was also removed. 40 epochs were averaged, and the radiofrequency evoked potential of interest was thus obtained.

The maxima and minima of the averaged signals were identified to determined latencies and amplitudes for each subject, in both arms and for each channel. Then, both the average and the standard deviation were calculated to obtain descriptive statistics of latency and amplitude of the signals.

### Example 3—Results

### 3.1. Experimental Session

The experimental session was carried out in 7 subjects, 6 men and a woman, of between 25 and 40 years old, which complied with the established requisites. The total duration of each session was of approximately 40 minutes, wherein the stimulation in each arm lasted, as a maximum, 8 minutes, and the remaining time was used for the preparation of the subject and the acquisition system.

The subjects reported that the perceived sensation, most of the times, was similar to a sting, while in some isolated situations the subjects reported tingling sensations similar to electrical stimulations. The sensations varied in relation to the zone wherein the stimulation tips were placed.

After the stimulations, small red areas were observed in the skin, of approximately 3 mm diameter. No pain was reported related thereto, while sometimes they were reported to generate a slight itch. These marks generally disappeared after a few hours, lasting up until two days in some subjects.

### 3.2. EEG Signals

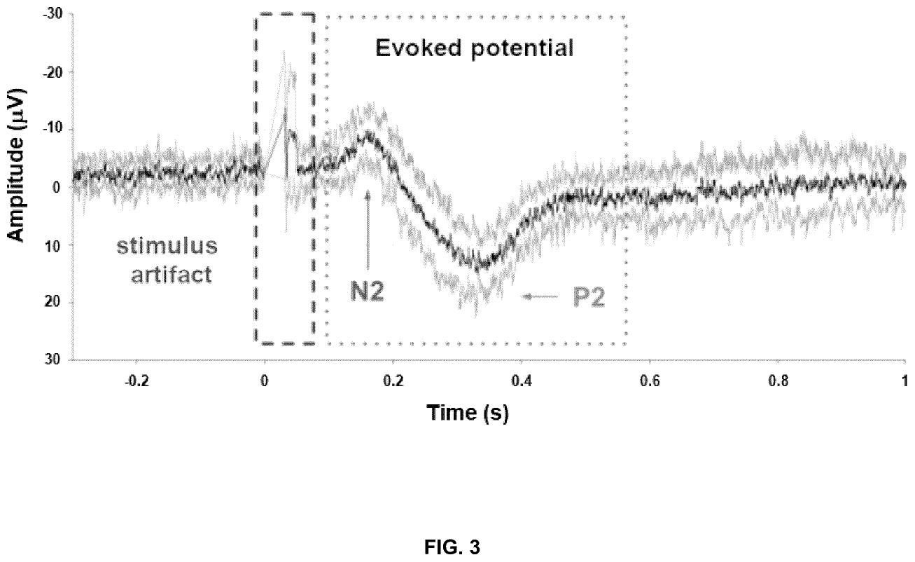

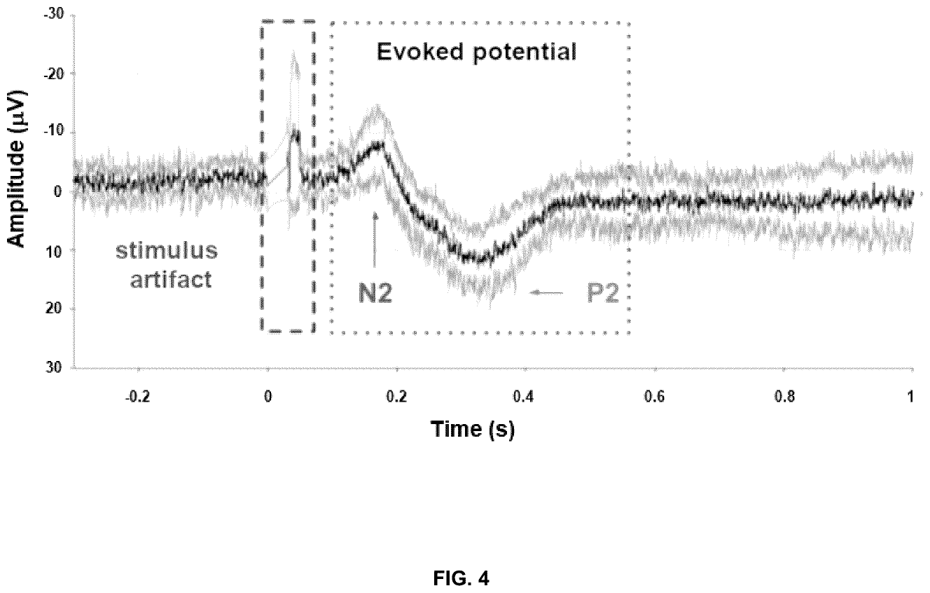

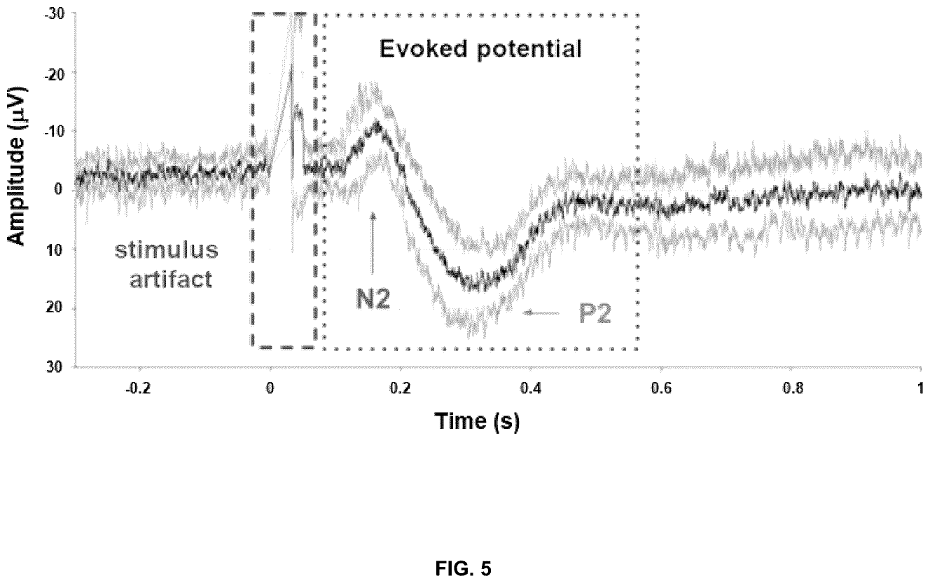

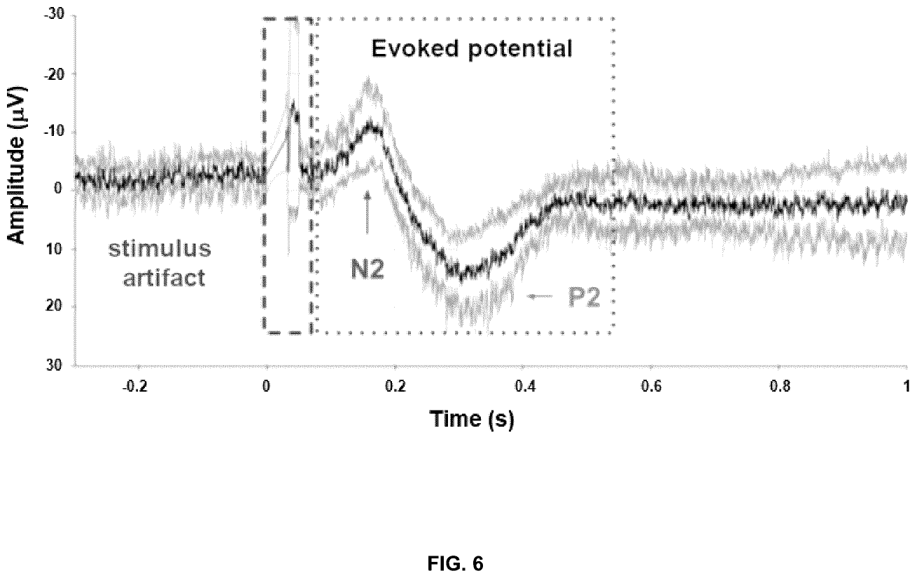

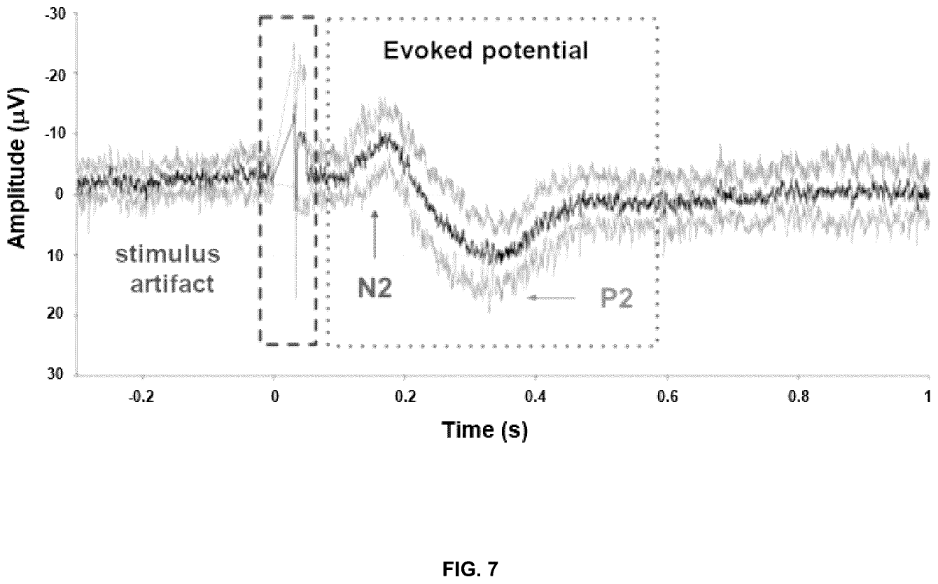

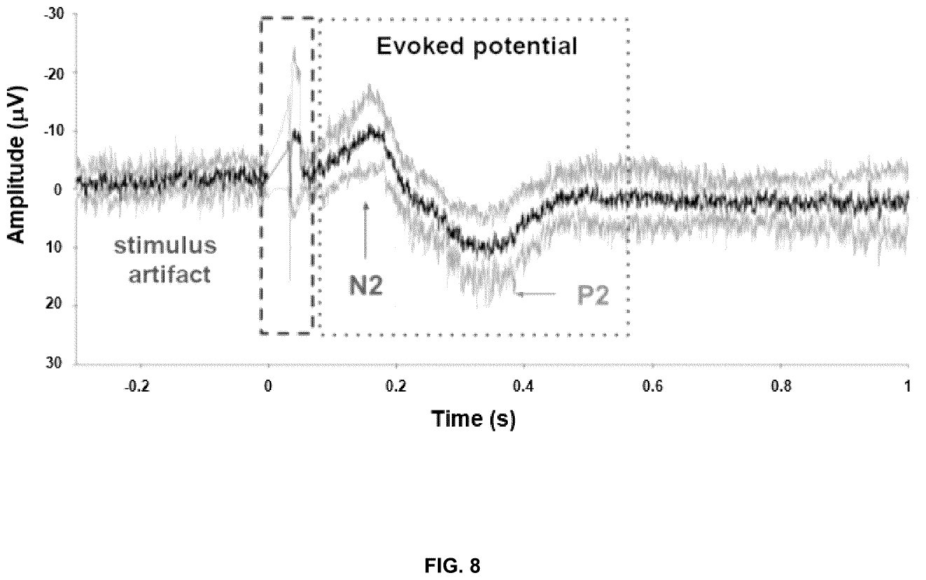

FIGS. 3 to 8 show the average and standard deviation of the processed signals of every subject for each arm, representing the C3, Cz and C4 channels with respect to the reference. These results were used to analyze the inter-subject variability.

The signals correspond to evoked potentials, wherein a biphasic N2-P2 signal (negative and positive peak) may be identified, as well as the stimulus artifact. Table 1 below shows the average and standard deviation values for latencies and amplitudes corresponding to each channel for each arm.

TABLE 1
Latency and amplitude values of the N2 −
P2 complex of the different channels for each arm

| | | N2 Latency (ms) | P2 Latency (ms) | Amplitud N2 − P2 (μV) |
|---|---|---|---|---|
| Arm | Channel | average ± SD | average ± SD | average ± SD |
| Right | C3 | 168.0 ± 15.7 | 328.3 ± 19.3 | 24.0 ± 4.3 |
| | Cz | 167.9 ± 18.3 | 304.9 ± 22.2 | 29.7 ± 6.4 |
| | C4 | 171.4 ± 13.9 | 325.9 ± 23.3 | 21.1 ± 4.9 |
| Left | C3 | 165.4 ± 15.9 | 317.9 ± 22.1 | 21.3 ± 5.7 |
| | Cz | 163.1 ± 12.4 | 316.1 ± 20.6 | 25.1 ± 10.3 |
| | C4 | 159.6 ± 13.8 | 332.6 ± 31.1 | 21.0 ± 6.6 |

3.3. Comparison with Other Stimulation Techniques

The evoked potentials generated by means of the different stimulation methods of the state of the art exhibit latency and amplitude values within a wide range, depending on the used equipment, the stimulation parameters, the experimental protocol, the stimulation zones and the anthropometrical features of the volunteers, among others.

Table 2 shows the normative latency and amplitude values in the back of the right hand for laser stimulation (CO₂ and Nd: YAP [14]) and for heat by contact in the right forearm [15]. Latency and amplitude values for electric stimulation have not yet been normed, but data in the back of the right hand for application with planar concentric and concentric needle electrodes have been reported. The values obtained with RF of channel Cz for the right arm are also included.

TABLE 2
Latency and amplitude values of the N2 − P2 complex of the different stimulation methods

| | | N2 Latency (ms) | P2 Latency (ms) | Amplitud N2 − P2 (μV) |
|---|---|---|---|---|
| Stimulation | | average ± SD | average ± SD | average ± SD |
| Laser | CO₂ | 236.0 ± 18.0 | 315.4 ± 23.1 | 18.3 ± 8.5 |
| | Nd:YAP | 225.0 ± 25.0 | 345.0 ± 33.0 | 27.0 ± 4.4 |
| Heat by contact | | 467.0 ± 43.1 | 591.3 ± 90.3 | 23.8 ± 14.9 |
| Electrical | Planar concentric | 157.8 ± 26.9 | 267.0 ± 39.0 | 24.8 ± 14.1 |
| intraepidermal | electrode | | | |
| | Concentric needle | 159.0 ± 39.0 | 340.0 ± 111.0 | 22.7 ± 8.4 |
| | electrode | | | |

| | | | |
|---|---|---|---|
| Radiofrequency | 167.9 ± 18.3 | 304.9 ± 22.2 | 29.7 ± 6.4 |

It may be appreciated that the latency of the N2 peak for RF is similar to the one for the electrical stimulation. This can be explained mainly because the duration and intensity of the stimuli, as well as the perceptual sensation reported, are very similar for both methods. Said latencies are slightly shorter than the ones reported for laser stimulation, probably because laser evoked potentials have a delay and jitter due to the heating and transduction effect. The latency of the P2 peak for RF has a value similar to the one obtained by laser stimulation. This could be due to the fact that P2 es a marked of early brain processing of pain, which may imply the attention and the assessment of the conductive context as integral components, and in both cases the beginning of the nociceptors activation is produced by heating of the tissue.

### REFERENCES

[1] Treede R-D, Lorenz J and Baumgartner U 2003 Clinical usefulness of laser-evoked potentials Neurophysiol. Clin. Neurophysiol. 33 303-14
[2] Valeriani M, Pazzaglia C, Cruccu G and Truini A 2012 Clinical usefulness of laser evoked potentials Neurophysiol. Clin. 42 345-53
[3] Hansen N, Kahn A K, Zeller D, Katsarava Z, Sommer C and Üçeyler N 2015 Amplitudes of pain-related evoked potentials are useful to detect small fiber involvement in painful mixed fiber neuropathies in addition to quantitative sensory testing—An electrophysiological study Front. Neurol. 6 1-10
[4] Omori S, Isose S, Misawa S, Watanabe K, Sekiguchi Y, Shibuya K, Beppu M, Amino H and Kuwabara S 2017 Pain-related evoked potentials after intraepidermal electrical stimulation to Aδ and C fibers in patients with neuropathic pain Neurosci. Res. 121 43-8
[5] Lefaucheur J P, Wahab A, Planté-Bordeneuve V, Sène D, Ménard-Lefaucheur I, Rouie D, Tebbal D, Salhi H, Créange A, Zouari H and Ng Wing Tin S 2015 Diagnosis of small fiber neuropathy: A comparative study of five neurophysiological tests Neurophysiol. Clin. 45 445-55
[6] Treede R-D, Kief S, Hölzer T and Bromm B 1988 Late somatosensory evoked cerebral potentials in response to cutaneous heat stimuli Electroencephalogr. Clin. Neurophysiol. 70 429-41
[7] Mouraux A, lannetti G D and Plaghki L 2010 Low intensity intra-epidermal electrical stimulation can activate Aδ-nociceptors selectively Pain 150 199-207
[8] S. -E. P and H. E 2010 A review on experimental assessments of pain threshold in healthy human subjects Basic Clin. Neurosci. 1 62-7
[9] Moloney N A, Hall T M and Doody C M 2012 Reliability of thermal quantitative sensory testing: A systematic review J. Rehabil. Res. Dev. 49 191-208
[10] Lelic D, Mørch C D, Hennings K, Andersen O K and M. Drewes A 2012 Differences in perception and brain activation following stimulation by large versus small area cutaneous surface electrodes Eur. J. Pain (United Kingdom) 16 827-37
[11] Inui K, Tran T D, Hoshiyama M and Kakigi R 2002 Preferential stimulation of Aδ fibers by intra-epidermal needle electrode in humans Pain 96 247-52
[12] Inui K and Kakigi R 2012 Pain perception in humans: use of intraepidermal electrical stimulation J. Neurol. Neurosurg. Psychiatry 83 551-6
[13] Truini A, Galeotti F, Romaniello A, Virtuoso M, lannetti G D & Cruccu G 2005. Laser-evoked potentials: normative values. Clin. Neurophysiol. 116 (4), 821-826.
[14] Di Stefano, G. et al. Diagnostic accuracy of laser-evoked potentials in diabetic neuropathy. Pain 158, 1100-1107 (2017).
[15] Granovsky, Y. et al. Normative data for Aδ contact heat evoked potentials in adult population. PAIN 157, 1156-1163 (2016)
[16] de Tommaso, M. et al. A comparative study of cortical responses evoked by transcutaneous electrical vs CO(2) laser stimulation. Clin. Neurophysiol. 122, 2482-2487 (2011)
[17] Mouraux, A, lannetti, G. D. & Plaghki, L. Low intensity intra-epidermal electrical stimulation can activate Aδ-nociceptors selectively. Pain 150, 199-207 (2010)

1. A system for the assessment of small nerve fiber function, comprising
a) a radiofrequency (RF) generator configured to deliver radiofrequency (RF) currents;
b) small-area RF probes to apply RF currents to a subject's skin;
c) a monitoring module for recording body responses to the electromagnetic pulses applied by the RF probes; and
d) a microprocessor configured to: i) send stimulus features to the RF generator for converting them to thermal stimuli according to stimulation parameters selected by a user of the device, and ii) analyze the body responses recorded by the monitoring module to provide an assessment of the small nerve fiber function.

2. The system of claim 1, wherein the RF generator is configured to generate RF currents of a duration ranging from 5 to 500 ms.

3. The system of claim 1, wherein the currents generated by the RF generator have a waveform selected from the group consisting of square-wave, linear, exponential, and bounded exponential.

4. The system of claim 3, wherein the waveform of the RF currents is square-wave.

5. The system of claim 1, wherein the small-area probes are selected from contact probes and contactless probes.

6. The system of claim 5, wherein the contactless probes comprise a distance-adjusting mechanism.

7. The system of claim 1, wherein the body response recorded by the monitoring module is selected from the group consisting of radiofrequency evoked potentials from an electroencephalogram, reaction times from a push-button and subjective pain ratings from a visual analog scale.

8. The system of claim 7, wherein the monitoring module is configured to record radiofrequency evoked potentials from an electroencephalogram

9. A method for diagnosing small fiber neuropathy, the method comprising the steps of:
1) providing a radiofrequency (RF) generator configured to deliver radiofrequency (RF) currents;
2) applying small-area probes to a subject's skin, wherein the small-area probes are capable of delivering the electromagnetic pulses to the subject's skin;
3) varying the RF stimulation intensity generated by the RF generator by means of an up-down staircase algorithm until a subjective sensory detection threshold is detected, wherein the detection of the subjective sensory detection threshold implies recording body responses from the subject by means of a monitoring module; and
4) comparing the obtained subjective sensory detection threshold with reference population values.

10. The method of claim 9, wherein step 3) of the method comprises the sub steps of:
a. increasing the stimulation intensity until an upper threshold is reached; and
b. decreasing the stimulation intensity until a lower threshold is reached.

11. The method of claim 10, comprising a step 3′, which comprises repeating sub steps a. and b. of step 3) a limited number of times to obtain a more precise estimation of the subjective sensory detection threshold.

12. The method of claim 9, wherein the subjective sensory detection threshold is a pain threshold.

13. The method of claim 9, wherein the small-area probes are contact probes, and are placed on the subject's skin.

14. The method of claim 9, wherein the small-area probes are contactless probes, fixed at a determined distance from the subject's skin by means of a distance-adjusting mechanism.

15. The method of claim 14, wherein the contactless probes are fixed at a distance of about 1 mm from the subject's skin.

16. The method of claim 9, wherein the body responses from the subject recorded in step 3) are selected from the group consisting of brain evoked potentials from an electroencephalogram, reaction times from a push-button and subjective pain ratings from a visual analog scale.

17. The method of claim 16, wherein the recorded body responses are brain evoked potentials from an electroencephalogram.

18. A method for diagnosing small fiber neuropathy, the method comprising the steps of:
1) providing a radiofrequency (RF) generator configured to deliver radiofrequency (RF) currents;
2) applying small-area probes to a subject's skin, wherein the small-area probes are capable of delivering the electromagnetic pulses to the subject's skin;
3) varying the RF stimulation intensity generated by the RF generator by means of an up-down staircase algorithm until a subjective pain threshold is detected, wherein the detection of the subjective pain threshold implies recording brain evoked potentials from an electroencephalogram from the subject by means of a monitoring module, this step comprising the sub steps of:
a. increasing the stimulation intensity until an upper threshold is reached; and
b. decreasing the stimulation intensity until a lower threshold is reached;
4) repeating sub steps a. and b. of step 3) a limited number of times to obtain a more precise estimation of the subjective pain threshold;
5) selecting a suitable multiplier of the detection threshold, and delivering a number of consecutive radiofrequency stimuli at different spots in the application region on the subject's skin while recording the brain evoked potentials from the electroencephalographic signals obtained in the electroencephalogram;
6) detecting salient features from the brain evoked potentials by means of an automatic detection algorithm; and
7) comparing the detected features with reference population values.
